(19) United States
(12) Patent Application Publication
(10) Pub. No.: US 2006/0194874 A1
(43) Pub. Date: Aug. 31, 2006

(54) Use of propionyl l-carnitine for the preparation of a medicament for the treatment of glaucoma

(76) Inventors: Calvani Menotti, Roma (IT); Emilia Reda, Roma (IT)

Correspondence Address: NIXON & VANDERHYE, PC, 901 NORTH GLEBE ROAD, 11TH FLOOR, ARLINGTON, VA, 22203, US

(21) Appl. No.: 10/537,057

(22) PCT Filed: Sep. 23, 2003
(86) PCT No.: PCT/IT03/00566

§ 371 Date: Jun. 2, 2005

(30) Foreign Application Priority Data
Oct. 1, 2002 (IT) RM2002A000492

(51) Int. Cl. A61K 31/22; A61K 31/7024; A61K 31/513

(52) U.S. Cl. 514/546; 514/23; 514/269

(57) ABSTRACT

Propionyl L-carnitine is used for the treatment of glaucoma and is preferably administrated orally or ophthalmically. Propionyl L-carnitine exhibits an anti-aptoptotic action on the nerve cells of the eye.

The invention described herein relates to the use of propionyl L-carnitine for the preparation of a medicament for the treatment of glaucoma.

### BACKGROUND OF THE INVENTION

Glaucoma is the second most common cause of blindness world-wide and may present at different ages. Glaucoma is classified as congenital, open-angle and closed-angle according to the underlying causes responsible for the reduction in outflow of aqueous humour. Each category is then subdivided into primary and secondary forms (Coleman, A. L., Glaucoma, Lancet 1999; 354: 1803-10). The latter forms commonly manifest themselves in the course of exfoliation syndrome, pigment dispersion syndrome or neovascularisation.

The aim of glaucoma therapy is to prevent any further loss of vision and to avoid the disease having a negative impact on the patient's life. The current therapies available induce a reduction of ocular pressure of such a nature as to prevent further damage to the ocular nerve. In fact, progression of the damage is caused by changes in the optic nerve and visual field that are consistent with the loss of several ganglionic cells or axons.

The classes of drugs used in glaucoma therapy are:

cholinergic agonists for topical use (e.g. pilocarpine, carbachol), which increase the outflow of aqueous humour; their side effects consist in: increased bronchial secretion, vomiting, diarrhoea, increased myopia, ocular or supraciliary pain, reduced vision, and apnoea;

beta-adrenergic antagonists for topical use (e.g. timolol, carteolol, levobunolol, betaxolol), which reduce the production of aqueous humour; their side effects consists in: congestive heart failure, bronchospasm, bradycardia, depression, confusion, impotence, deterioration of myasthenia gravis, and increased blood cholesterol levels;

adrenergic agonists for topical use (e.g. epinephrine, apraclonidine, brimonidine), which reduce both the resistance to outflow and the production of aqueous humour; their side effects consist in: increased blood pressure, tachyarrhythmia, tremors, anxiety, headache, dilation of the pupils, and allergic reactions;

carbonic anhydrase inhibitors for topical or oral use (e.g. topical dorzolamide and brinzolamide; oral acetazolamide and metazolamide), which reduce the production of aqueous humour; their side effects consist in: malaise, anorexia, depression, paraesthesias, abnormal serum electrolytes, kidney stones, blood dyscrasia, allergic reactions, and taste alterations (bitter or acid taste sensations);

prostaglandin analogues (e.g. latanoprost), which increase the outflow of aqueous humour; their side effects consist in increased pigmentation of the iris and eyelashes, and hypertrichosis.

From what has been said so far it will be understood that the current glaucoma therapy is aimed mainly, if not exclusively, at controlling ocular pressure by means of an action on both the production and outflow of aqueous humour.

In glaucoma there is progressive degeneration of the ganglionic cells, related in some cases to apoptosis (N. Pescosolido, et al., Acta Ophthalmologica Scandinavica, 1988, Supplement 227, 20-21). Cell death by apoptosis is also accompanied by necrosis, no way of preventing which exists to date. Conversely, one may seek to produce an effect on the apoptotic phenomenon by effectively exerting neuroprotection (McKinnon, 1997, Curr. Op. Ophthalmol., 8: 28-37). As regards the glaucoma model based on the apoptosis mechanism, see the above-cited study by Pescosolido et al. and the Experimental Postgraduate Degree Thesis by Renata Rosa, Institute of Ophthalmology, “La Sapienza” University, Rome, Academic Year 2000-2001.

In U.S. Pat. No. 5,145,871, filed in the name of the present Applicant, a glaucoma treatment is described using acetyl D-carnitine, which is a different molecule from propionyl L-carnitine. In the above-mentioned patent, the preferred administration form is an eye-drop formulation. Acetyl D-carnitine exerts its curative action by lowering intraocular pressure.

U.S. Pat. No. 5,883,127, filed in the name of the present Applicant, describes the use of alkanoyl L-carnitine, where the alkanoyl group has 2 to 6 carbon atoms, for the preparation of a medicament useful for the treatment of retinopathies. Particularly indicated among the retinopathies are maculopathy, whether age-related or not, diabetic retinopathy, neuroretinopathy (Batten-Mayou disease, hereditary dominant drusen) and age-related macular degeneration. The diseases indicated in this patent are aetiopathogenetically unrelated to glaucoma.

There is therefore now a perceived need for a drug that acts on the phenomenon of apoptosis of the nerve cells, preserving them or at least delaying their death, and thus the ocular neurodegeneration. At the same time, there is also a strongly perceived need for a drug carrying reduced side effects.

### SUMMARY OF THE INVENTION

It has now surprisingly been found that propionyl L-carnitine exerts a protective effect on the ganglionic cells of the optic nerve, effectively preventing or at least delaying cellular apoptosis. Thus, propionyl L-carnitine (hereinafter also referred to as PLC) is suitable as an active ingredient for the preparation of a medicament for the treatment of glaucoma.

On the basis of the teaching of the prior art, it was not predictable that propionyl L-carnitine would exert an anti-apoptotic-type neuroprotective effect on the cells of the optic nerve.

Propionyl L-carnitine is known as a medicament for the treatment of disease of the vascular district and is marketed under the brand name of DROMOS®. Among the main indications for its use is intermittent claudication.

Thus, one object of the present invention is the use of propionyl L-carnitine or of one of its pharmaceutically acceptable salts for the preparation of a medicament useful for the treatment of glaucoma.

The invention will now be illustrated in detail also by means of examples and figures in which:

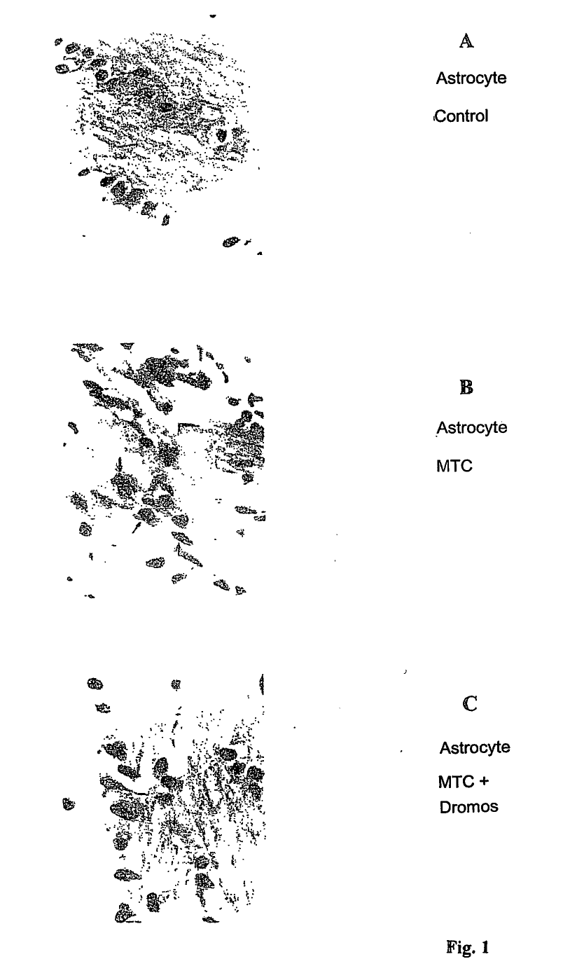

FIG. 1 shows semithin histological sections of the optic nerves of normal rats (FIG. 1A), of rats treated with methylcellulose (MTC) (FIG. 1B) and of rats treated with MTC and propionyl L-carnitine (FIG. 1C);

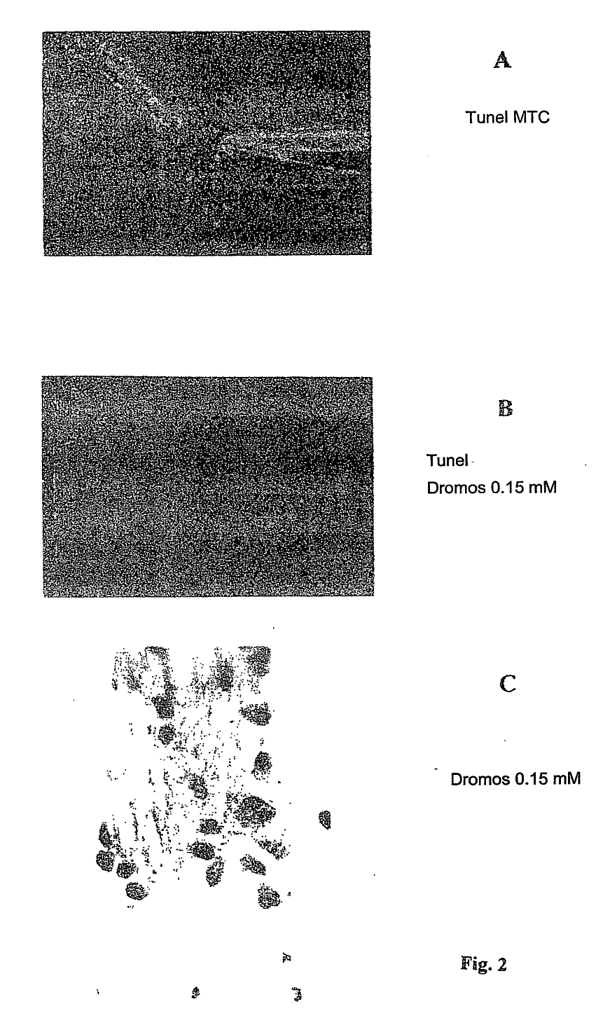

FIG. 2 shows semithin sections of rat optic nerve and retina treated with MTC, using fluorescence microscopy (TUNEL technique) to detect apoptotic cells (FIG. 2A) and treated with MTC and propionyl L-carnitine, indicating the absence of apoptotic appearances (FIG. 2B);

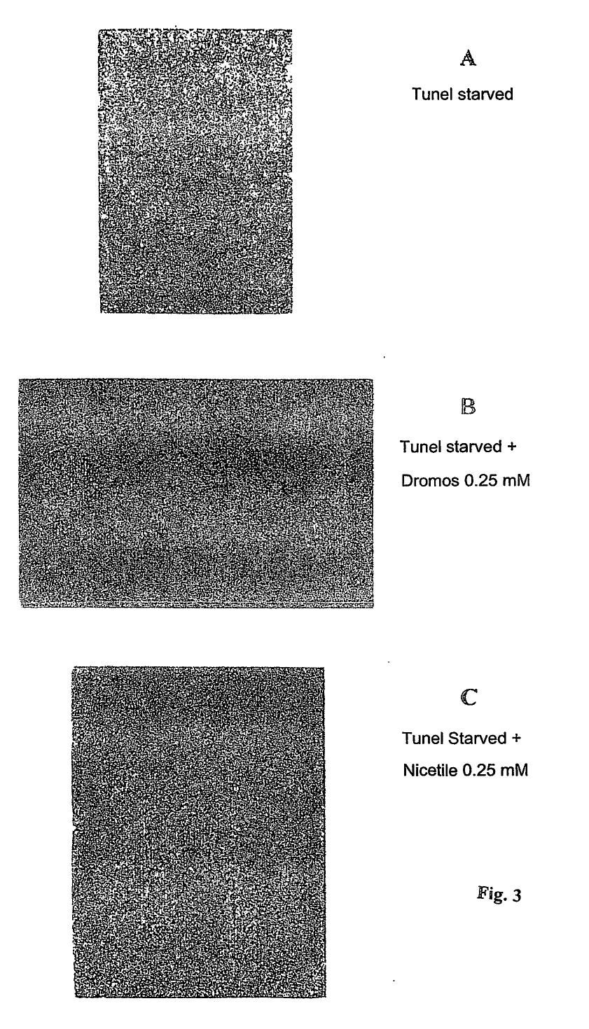

FIG. 3 shows chromatin status in cells taken as samples (FIG. 3A) and in cells treated with propionyl L-carnitine (FIG. 3B).

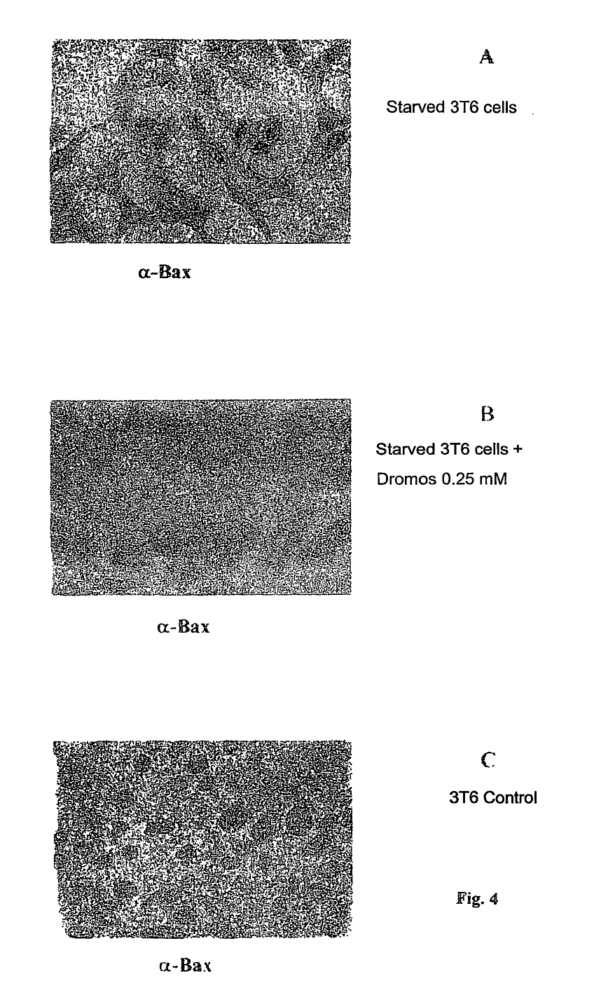

FIG. 4 illustrates the cytoimmunological localisation investigations on the type of cell death to which the serum-deprived cells are subjected (FIG. 4A); on serum-deprived 3T6 cells treated with propionyl L-carnitine (FIG. 4B), and the morphological reference aspect (control) (FIG. 4C).

### DETAILED DESCRIPTION OF THE INVENTION

For the purposes of the present invention, propionyl L-carnitine or one of its pharmaceutically acceptable salts will be suitably formulated in a conventional pharmaceutical composition.

This composition can be prepared according to the normal knowledge of the person skilled in this field, for example, by consulting the well-known “Remington's Pharmaceutical Sciences, Mack Publishing & Co.”.

Further examples of pharmaceutical compositions are to be found in U.S. Pat. Nos. 6,380,252, 6,346,282, 6,306,392 and 6,253,346, all filed in the name of the present Applicant or its subsidiary Sigma-Tau HealthScience S.p.A.

The medicament according to the present invention can be administered orally, parenterally or topically. The oral route is the preferred one for ease of use, but it can also be combined with the ophthalmic formulation, e.g. in the form of eye-drops. An oral formulation of propionyl L-carnitine is known commercially as DROMOS®.

The doses and posology will be decided by the primary care physician, according to his or her experience, the state of the disease and the patient's condition. Indicatively, a dose of 2 g/day of propionyl L-carnitine is preferred. The drug can be administered by mouth or in some other way that the physician may deem opportune. Advantageously, this dose can also be reached by combining oral administration, or administration by some other route, with topical optical administration, e.g. by means of eye-drops.

What is meant by a pharmaceutically acceptable salt of propionyl L-carnitine is any salt of the latter with an acid that does not give rise to unwanted toxic or side effects.

These acids are well known to pharmacologists and to experts in pharmaceutical technology.

Examples of such salts, though not exclusively these, are: chloride, bromide, orotate, acid aspartate, acid citrate, magnesium citrate, acid phosphate, fumarate and acid fumarate, magnesium fumarate, lactate, maleate and acid maleate, mucate, acid oxalate, pamoate, acid pamoate, acid sulphate, glucose phosphate, tartrate, acid tartrate, magnesium tartrate, 2-amino-ethane sulphonate, magnesium 2-amino-ethane sulphonate, choline tartrate, and trichloroacetate.

The following examples further illustrate the invention.

### EXAMPLE 1

In-vivo Anti-Apoptotic Effect of Propionyl L-Carnitine

The effect of propionyl L-carnitine was evaluated in an in-vivo experimental rat model. This model of induction of programmed cell death (apoptosis) and/or necrosis of ganglionic cells of the retina and/or astrocytes of the optic nerve is described in the above-mentioned Acta Ophthalmologica Scandinavica, 1988, Supplement 27, 20-21 and Experimental Postgraduate Degree Thesis, Institute of Ophthalmology, “La Sapienza” University, Rome. Intraocular hypertension was induced by means of injecting methylcellulose (MTC) into the anterior chamber. The rats were divided into three groups: 1) untreated (blanks), 2) rats treated with 2% MTC (10 μL), and 3) rats treated with MTC and propionyl L-carnitine 0.15 mM final concentration injected into the anterior chamber. In semithin histological sections of untreated rat optic nerve (haematoxylin-eosin staining), normal longitudinal organisation of the astrocytes among the fibres was detected (FIG. 1A). In contrast, in rats treated with MTC, loss of cell organisation and the presence of necrotic astrocytes were detected (FIG. 1B). In the group treated with MTC and propionyl L-carnitine, the astrocytes presented longitudinal organisation among the fibres and no necrotic cells were detected, thus demonstrating the protective effect of propionyl L-carnitine (FIG. 1C).

In semithin sections of the optic nerve and retina of rats treated with MTC, using fluorescence microscopy (TUNEL method) to detect apoptotic cells, diffuse staining was observed in the various layers of the retina and optic nerve, indicating the presence of numerous cells undergoing apoptosis (FIG. 2A). In the optic nerves of rats treated with MTC and propionyl L-carnitine, the presence of TUNEL fluorescence was not observed, indicating the absence of apoptotic appearances (FIG. 2B).

These results show that propionyl L-carnitine protects the optic nerve and the retinal cells against necrosis and apoptosis due to ocular hypertension (experimental glaucoma).

### EXAMPLE 2

Materials and Methods

3T6 cells (murine fibroblast line) grown in complete medium (DMEM: 10% NBS, 2% PEST and 2% glutamine) were treated with propionyl L-carnitine 24 hours after plating the cells. 48 hours after plating (24 hours after administration of the substance), a cell count was performed on the cells thus treated with an erythrosine stain that detects cells that are no longer viable (diluted 1:5 with PBS A buffer) from which the cell survival data were obtained. As controls, we used cells cultivated in complete medium on which the cell count was performed, again 24 hours after plating.

In subsequent experiments, apoptosis was induced in 3T6 cells 24 hours after plating by means of serum deprivation, and simultaneously propionyl L-carnitine was added to the cells. Three different concentrations of the compound were used: 0.25 mM, 0.55 mM and 1.1 mM. 48 hours after plating (24 hours after administration of the substance) a cell count with erythrosine staining was performed. Cells grown in complete medium were taken as the negative control and serum-deprived cells as the positive control.

To assess the chromatin status (and thus the apoptosis) in serum-deprived 3T6 cells, and, in cells treated with propionyl L-carnitine, a TUNEL (terminal deoxynucleotidyl transferase-mediated dUTP-biotin end labelling) reaction was performed in vitro. TUNEL labelling is a technique that consists in the addition of nucleotides (conjugated with fluorescein) at the free 3′-OH extremities present in the fragmented DNA. This reaction is catalysed by the terminal enzyme transferase. The TUNEL technique was performed on untreated serum-deprived cells and on serum-deprived cells to which the three concentrations (0.25 mM, 0.55 mM, and 1.1 mM) of propionyl L-carnitine already used for the cell counts were administered. By way of confirmation of what was observed with the TUNEL technique, a cytoimmunological localisation reaction was run on the samples with a primary monoclonal antibody targeted against BAX (a protein known in the literature as being present in apoptotic cells). The binding of the primary antibody with its epitope to BAX was detected with a secondary antibody that exploits DAB (diaminobenzene) staining. The cells subjected to cytoimmunological localisation were deprived of serum 24 hours after plating and treated with propionyl L-carnitine at the above-mentioned concentrations. Cells grown in complete medium were used as the positive control, while the negative control was prepared with cells deprived of serum 24 hours after plating.

Results

The data obtained by counting the cells grown in complete medium to which propionyl L-carnitine was added show an increase in cell growth as compared to the control cells not treated with the compound. The experimental evidence suggests that propionyl L-carnitine is probably a cell trophism and growth factor. The cell counts performed on serum-deprived cells treated with the compound demonstrated the protective action of the substance examined against serum deprivation. In effect, the results obtained show a reduction in cell mortality compared to the values with the serum-deprived cells taken as the positive control, though the cell viability is still less than that obtained in the negative control.

With the TUNEL technique, as illustrated above, we carried out a study of chromatin status. The data obtained indicated that the untreated serum-deprived cells undergo apoptosis, a phenomenon clearly demonstrated in this case by the fluorescence present in the cells taken as a sample (FIG. 3A). In contrast, the cells treated with propionyl L-carnitine showed a marked reduction in chromatin fragmentation, observed thanks to a reduced intensity of the fluorescence phenomenon (FIG. 3B).

Furthermore, the three concentrations of the substance assayed revealed a cytoprotective effect on the samples considered which increased with the increase in concentration (0.25 mM, 0.55 mM, 1.1 mM).

Cytoimmunological localisation investigations provided further indications as to the type of cell death the serum-deprived cells were subject to (FIG. 4A). These data show that the serum-deprived cells die as a result of BAX-dependent apoptosis. This finding was revealed by the cytoplasmic localisation of BAX, a protein that takes part in the cascade of cell events leading to apoptosis. In the literature it is known that the family of the caspases also take part in the apoptotic mechanism; caspases are proteases to serine and BCL-2, an anti-apoptotic protein that appears to exert an antagonist action on BAX. The cytoimmunological localisation experiment was also performed on serum-deprived 3T6 cells treated with propionyl L-carnitine, revealing, in this case, a lower expression of BAX compared to that observed in the untreated serum-deprived cells (FIG. 4B). FIG. 4C shows the morphological reference aspect (control).

The staining thus obtained not only provided qualitative data, revealing the presence of absence of BAX, but also yielded quantitative findings, making it possible to detect the increase or decrease in expression of the protein as a result of treatment of the samples with propionyl L-carnitine. This expression proved to be proportional to the different concentrations of the substance assayed.

Counterstaining with a nuclear dye such as haematoxylin was also performed in order to confirm the fact that the staining obtained with cytoimmunological localisation was effectively cytoplasmic. On the basis of the data reported in the literature, in fact, it is known that BAX is a cytoplasmically localised protein. In addition, this staining provides information as to the state of chromatin condensation, revealing very substantial alterations in serum-deprived cells. These alterations tend to be reduced after treatment with propionyl L-carnitine. On comparing the data obtained in the cell counts, with the TUNEL technique and with cytoimmunological localisation, an improvement can be found in cell survival after treatment with propionyl L-carnitine. This proves evident both in cells grown in complete medium and in cells grown in conditions in which apoptosis is induced as a result of serum deprivation. Since it is known that as a result of glaucoma retinal ganglionic cells die by apoptosis, the reduction of this phenomenon, due to propionyl L-carnitine, demonstrates the validity of its therapeutic application in glaucoma sufferers.

### EXAMPLE 3

Clinical Trial

Ten patients suffering from glaucoma were treated with propionyl L-carnitine (DROMOS®) by mouth at a dose of 2 g/day for periods ranging from 15 to 50 days. Ocular arterial flow was studied by means of ocular colour Doppler ultrasonography. The posterior (nasal) ciliary artery, the posterior (temporal) ciliary artery, the central retinal and ophthalmic arteries were examined. Systolic and diastolic flow rates were expressed in cm/sec; the ratio (systolic—diastolic flow)/systolic flow expresses the peripheral resistance index. Visual fields before and after treatment with DROMOS® were compared.

Patients suffering from glaucoma show a reduction in arterial flow in the various vessels, with a marked increase in ocular peripheral resistance, and this picture, if protracted over time, leads to damage to the optic nerve.

Treatment with DROMOS® increases the flows and reduces the resistance index in the sectors affected. Intraocular pressure is not affected by the treatment, and, in fact, there is no significant reduction in IOP.

By way of examples, two cases are reported:

Patient 1

Age: 80 years

Male

Intraocular pressure<18

Diagnosis: glaucoma; duration of treatment: 30 days

Visual field (Octopus) before treatment: 11.6 OS MS; MD 2

Visual field (Octopus) after treatment; 20.2 OS MS; MD 5.6

Ocular Colour Doppler US

Before treatment:

Posterior nasal ciliary artery: SN 5.96/2.21 (R.I. 0.63) systolic flow/diastolic flow cm/sec

After treatment:

Posterior nasal ciliary artery: SN 16.7/5.75 (R.I. 0.66).

Patient 2

Age; 66 years

Female

Intraocular pressure<18

Diagnosis: open-angle glaucoma (OAG); duration of treatment: 12 days

Visual field (Octopus) before treatment: OD MS15.5; MD 11.1

Visual field (Octopus) after treatment: OD MS 16.2; MD 8.4

Ocular Colour Doppler US

Before treatment:

Posterior temporal ciliary artery: SN 5.75/1.92 (R.I. 0.67)

After treatment:

Posterior temporal ciliary artery: DX 8.65/3.86 (R.I. 0.55).

The results show that propionyl L-carnitine, at the preferred dose of 2 g/day by mouth, improves the results in terms of ocular vessel flows and also improves visual fields in subjects with stable glaucoma. Propionyl L-carnitine exerts its therapeutic action by protecting perfusion of the optic nerve and of the retinal structures.

1. A method of treating glaucoma comprising administering to a subject an effective amount of propionyl L-carnitine or one of its pharmaceutically acceptable salts.

2. The method according to claim 1, where said treatment is administered orally, parenterally or ophthalmically.

3. The method according to claim 2, where said treatment is administered in a combined oral and ophthalmic formulation.

4. The method according to claim 1, where 2 g/day of propionyl L-carnitine is admnistered.

5. The method according to claim 1, wherein said salt of propionyl L-carnitine is selected from the group consisting of chloride, bromide, orotate, acid aspartate, acid citrate, magnesium citrate, acid phosphate, fumarate and acid fumarate, magnesium fumarate, lactate, maleate and acid maleate, mucate, acid oxalate, pamoate, acid pamoate, acid sulphate, glucose phosphate, tartrate, acid tartrate, magnesium tartrate, 2-amino-ethane
